(19) United States
(12) Patent Application Publication
(10) Pub. No.: US 2024/0033085 A1
(43) Pub. Date: Feb. 1, 2024

(54) METHOD FOR TRAVERSING AN ANATOMICAL VESSEL WALL

(71) Applicant: MVRX, Inc., San Mateo, CA (US)

(72) Inventors: Patrick P. Wu, San Carlos, CA (US); David A. Rahdert, San Mateo, CA (US); Richard T. Childs, San Mateo, CA (US); David R. Tholfsen, San Leandro, CA (US)

(21) Appl. No.: 18/038,647

(22) PCT Filed: Dec. 7, 2021
(86) PCT No.: PCT/US2021/062198

§ 371 Date: May 24, 2023

Related U.S. Application Data
(60) Provisional application No. 63/122,843, filed on Dec. 8, 2020.

(51) Int. Cl. A61F 2/24; A61B 17/00; A61M 25/09

(52) U.S. Cl. CPC A61F 2/2466; A61B 17/00234; A61M 25/09; A61F 2250/0007; A61B 2017/00243; A61M 2025/09183

(57) ABSTRACT

A method for traversing an anatomical vessel wall of a subject is provided. The invention allows the crossing of a wire from one anatomical lumen, such as an artery, vein, esophagus, intestine or airway, through tissue, into another anatomical lumen, or cavity, or into a solid mass of tissue. In some aspects, the invention allows the crossing of a wire from the greater cardiac vein (GCV) into the left atrium without relying on another device in the left atrium to facilitate the crossing.

### CROSS-REFERENCE TO RELATED APPLICATION(S)

This application claims benefit of priority under 35 U.S.C. § 119(e) of U.S. Provisional Patent Application Ser. No. 63/122,843, filed Dec. 8, 2020. The disclosure of the prior application is considered part of and is incorporated by reference in the disclosure of this application.

### BACKGROUND OF THE INVENTION

### Field of the Invention

The present invention relates generally to medical procedures, and more particularly to a method for traversing an anatomical vessel wall of a subject during surgical procedures, including those for treatment of cardiac disorders.

### Background Information

Treatments for mitral valve regurgitation are widely varied, encompassing both replacement valves, as well as a number of approaches that facilitate repair and reshaping of the valve by use of an implant. While many such approaches rely on intravascular delivery of an implant, these often utilize a system of multiple catheters that are repeatedly exchanged, which is an often complex and time-consuming process. To appreciate the difficulties and challenges associated with delivery and deployment of an implant within the human heart, it is useful to understand various aspects of the anatomy of the heart as well as conventional methods of deploying an implant for treatment of mitral valve regurgitation.

The Anatomy of a Healthy Heart

As can be seen in FIG. 2A, the human heart is a double-sided (left and right side), self-adjusting pump, the parts of which work in unison to propel blood to all parts of the body. The right side of the heart receives poorly oxygenated (“venous”) blood from the body from the superior vena cava and inferior vena cava and pumps it through the pulmonary artery to the lungs for oxygenation. The left side receives well-oxygenation (“arterial”) blood from the lungs through the pulmonary veins and pumps it into the aorta for distribution to the body.

The heart has four chambers, two on each side; the right and left atria, and the right and left ventricles. The atriums are the blood-receiving chambers, which pump blood into the ventricles. The ventricles are the blood-discharging chambers. A wall composed of fibrous and muscular parts, called the interatrial septum separates the right and left atriums (see FIGS. 2B-2D). An anatomic landmark on the interatrial septum is an oval, thumbprint sized depression called the oval fossa, or fossa ovalis (FO), shown in FIG. 2C, which is a remnant of the oval foramen and its valve in the fetus and thus is free of any vital structures such as valve structure, blood vessels and conduction pathways. The synchronous pumping actions of the left and right sides of the heart constitute the cardiac cycle. The cycle begins with a period of ventricular relaxation, called ventricular diastole. The cycle ends with a period of ventricular contraction, called ventricular systole 3. The heart has four valves (see FIGS. 2B and 2C) that ensure that blood does not flow in the wrong direction during the cardiac cycle; that is, to ensure that the blood does not back flow from the ventricles into the corresponding atria, or back flow from the arteries into the corresponding ventricles. The valve between the left atrium and the left ventricle is the mitral valve. The valve between the right atrium and the right ventricle is the tricuspid valve. The pulmonary valve is at the opening of the pulmonary artery. The aortic valve is at the opening of the aorta.

At the beginning of ventricular diastole (ventricular filling), the aortic and pulmonary valves are closed to prevent back flow from the arteries into the ventricles.

Shortly thereafter, the tricuspid and mitral valves open, as shown in FIG. 2B, to allow flow from the atriums into the corresponding ventricles. Shortly after ventricular systole (ventricular emptying) begins, the tricuspid and mitral valves close, as shown in FIG. 2C, to prevent back flow from the ventricles into the corresponding atriums, and the aortic and pulmonary valves open to permit discharge of blood into the arteries from the corresponding ventricles.

The opening and closing of heart valves occur primarily as a result of pressure differences. For example, the opening and closing of the mitral valve occurs as a result of the pressure differences between the left atrium and the left ventricle. During ventricular diastole, when ventricles are relaxed, the venous return of blood from the pulmonary veins into the left atrium causes the pressure in the atrium to exceed that in the ventricle. As a result, the mitral valve opens, allowing blood to enter the ventricle. As the ventricle contracts during ventricular systole, the intraventricular pressure rises above the pressure in the atrium and pushes the mitral valve shut.

As FIGS. 2B-2C show, the anterior (A) portion of the mitral valve annulus is intimate with the non-coronary leaflet of the aortic valve. Notably, the mitral valve annulus is near other critical heart structures, such as the circumflex branch of the left coronary artery (which supplies the left atrium, a variable amount of the left ventricle, and in many people the SA node) and the AV node (which, with the SA node, coordinates the cardiac cycle). In the vicinity of the posterior (P) mitral valve annulus is the coronary sinus and its tributaries. These vessels drain the areas of the heart supplied by the left coronary artery. The coronary sinus and its tributaries receive approximately 85% of coronary venous blood. The coronary sinus empties into the posterior of the right atrium, anterior and inferior to the fossa ovalis, as can be seen FIG. 2C. A tributary of the coronary sinus is called the great cardiac vein, which courses parallel to the majority of the posterior mitral valve annulus, and is superior to the posterior mitral valve annulus by an average distance of about 9.64+/−3.15 millimeters.

Characteristics and Causes of Mitral Valve Dysfunction

When the left ventricle contracts after filling with blood from the left atrium, the walls of the ventricle move inward and release some of the tension from the papillary muscle and chords. The blood pushed up against the under-surface of the mitral leaflets causes them to rise toward the annulus plane of the mitral valve. As they progress toward the annulus, the leading edges of the anterior and posterior leaflet come together forming a seal and closing the valve. In the healthy heart, leaflet coaptation occurs near the plane of the mitral annulus. The blood continues to be pressurized in the left ventricle until it is ejected into the aorta. Contraction of the papillary muscles is simultaneous with the contraction of the ventricle and serves to keep healthy valve leaflets tightly shut at peak contraction pressures exerted by the ventricle.

In a healthy heart (shown in FIGS. 2E-2F), the dimensions of the mitral valve annulus create an anatomic shape and tension such that the leaflets coapt, forming a tight junction, at peak contraction pressures. Where the leaflets coapt at the opposing medial (CM) and lateral (CL) sides of the annulus are called the leaflet commissures. Valve malfunction can result from the chordae tendineae (the chords) becoming stretched, and in some cases tearing. When a chord tears, the result is a leaflet that flails. Also, a normally structured valve may not function properly because of an enlargement of or shape change in the valve annulus. This condition is referred to as a dilation of the annulus and generally results from heart muscle failure. In addition, the valve may be defective at birth or because of an acquired disease. Regardless of the cause, mitral valve dysfunction can occur when the leaflets do not coapt at peak contraction pressures, as shown in FIG. 2G. In such cases, the coaptation line of the two leaflets is not tight at ventricular systole. As a result, an undesired back flow of blood from the left ventricle into the left atrium can occur, commonly known as mitral regurgitation. This has two important consequences. First, blood flowing back into the atrium may cause high atrial pressure and reduce the flow of blood into the left atrium from the lungs. As blood backs up into the pulmonary system, fluid leaks into the lungs and causes pulmonary edema. Second, the blood volume going to the atrium reduces volume of blood going forward into the aorta causing low cardiac output. Excess blood in the atrium over-fills the ventricle during each cardiac cycle and causes volume overload in the left ventricle.

Mitral regurgitation is categorized into two main types: i) organic or structural; and ii) functional. Organic mitral regurgitation results from a structurally abnormal valve component that causes a valve leaflet to leak during systole. Functional mitral regurgitation results from annulus dilation due to primary congestive heart failure, which is itself generally surgically untreatable, and not due to a cause like severe irreversible ischemia or primary valvular heart disease. Organic mitral regurgitation is seen when a disruption of the seal occurs at the free leading edge of the leaflet due to a ruptured chord or papillary muscle making the leaflet flail; or if the leaflet tissue is redundant, the valves may prolapse the level at which coaptation occurs higher into the atrium with further prolapse opening the valve higher in the atrium during ventricular systole. Functional mitral regurgitation occurs as a result of dilation of heart and mitral annulus secondary to heart failure, most often as a result of coronary artery disease or idiopathic dilated cardiomyopathy. Comparing a healthy annulus to an unhealthy annulus, the unhealthy annulus is dilated and, in particular, the anterior-to-posterior distance along the minor axis (line P-A) is increased. As a result, the shape and tension defined by the annulus becomes less oval and more round. This condition is called dilation. When the annulus is dilated, the shape and tension conducive for coaptation at peak contraction pressures progressively deteriorate.

Prior Treatment Modalities

It is reported that twenty-five percent of the six million Americans who will have congestive heart failure will have functional mitral regurgitation to some degree. This constitutes the 1.5 million people with functional mitral regurgitation. In the treatment of mitral valve regurgitation, diuretics and/or vasodilators can be used to help reduce the amount of blood flowing back into the left atrium. An intra-aortic balloon counterpulsation device is used if the condition is not stabilized with medications. For chronic or acute mitral valve regurgitation, surgery to repair or replace the mitral valve is often necessary.

By interrupting the cycle of progressive functional mitral regurgitation, it has been shown in surgical patients that survival is increased and in fact forward ejection fraction increases in many patients. The problem with surgical therapy is the significant insult it imposes on these chronically ill patients with high morbidity and mortality rates associated with surgical repair.

Currently, patient selection criteria for mitral valve surgery are very selective and typically performed only on patients having normal ventricular function, generally good health, a predicted lifespan of greater than 3 to 5 years, NYHA Class III or IV symptoms, and at least Grade 3 regurgitation. Patients that do not meet these requirements, typically older patients in poor health, are not good candidates for surgical procedures, especially open surgical procedures. Such patients benefit greatly from shorter, less invasive surgical procedures that improve valve function. However, such patients could benefit from further improvements in minimally invasive surgical procedures to deploy such valve treatment and repair implants, systems, reducing the complexity of delivery systems and duration of the procedures, as well as consistency, reliability and ease of use.

Thus, there is a need for further improvements that reduce the complexity of such delivery systems and improved methods of delivery that reduce the duration of the procedures, and improve the consistency, reliability and ease of use for the clinician in the deployment of heart implants for treatment of mitral valve regurgitation.

### SUMMARY OF THE INVENTION

The present invention provides a method for traversing an anatomical vessel wall of a subject. In various aspects, the invention allows the crossing of a wire from one anatomical lumen, such as an artery, vein, esophagus, intestine or airway, through tissue, into another anatomical lumen, or cavity, or into a solid mass of tissue. In some aspects, the invention allows the crossing of a wire from the greater cardiac vein (GCV) into the left atrium without relying on another device in the left atrium to facilitate the crossing.

Accordingly, in one embodiment, the invention provides a method for traversing a vessel wall. The method includes: advancing a catheter into a first anatomical lumen having a vessel wall to a first location, the catheter having a lumen extending along a length of the catheter, a distally disposed opening, and a stabilizing element; stabilizing the catheter within the first lumen via the stabilizing element at the first location; advancing a penetrating guidewire along the lumen of the catheter toward the distally disposed opening to the first location, wherein the penetrating guidewire comprises a tip, the tip having shape memory and configured to form a capture structure upon crossing the vessel wall; and penetrating the vessel wall by advancing the penetrating guidewire out of the distally disposed opening and traversing the vessel wall into a second anatomical lumen or tissue, thereby traversing the vessel wall.

In another embodiment, the invention provides a method of treating mitral valve regurgitation in a subject by reshaping a heart chamber of a subject. The method includes inserting, through a vascular access site, a catheter, and advancing the catheter along a first anatomical lumen having a vessel wall to a first location proximate a heart of the subject, the catheter having a lumen extending along a length of the catheter, a distally disposed opening, and a stabilizing element; stabilizing the catheter within the first lumen via the stabilizing element at the first location; advancing a penetrating guidewire along the lumen of the catheter toward the distally disposed opening to the first location; penetrating the vessel wall by advancing the penetrating guidewire out of the distally disposed opening and traversing the vessel wall into a heart chamber, wherein the penetrating guidewire comprises a tip, the tip having shape memory and configured to form a capture structure upon crossing the vessel wall; advancing a first anchor to the first location via the lumen of the catheter, wherein the first anchor is coupled to the first anchor at a first end of the bridging element; advancing a second end of the bridging element through the penetrated vessel wall at the first location; advancing a second anchor along the bridging element and deploying the second anchor at a second location in or proximate the heart, the bridging element spanning across the heart chamber; and shortening a length of the bridging element thereby reshaping the chamber of the heart and coupling the second end of the bridging element to the deployed second anchor while the chamber of the heart is reshaped so that the chamber of the heart remains reshaped, thereby treating mitral valve regurgitation in the subject.

### BRIEF DESCRIPTION OF THE FIGURES

FIG. 1 depicts an overview of a conventional catheter system for intravascular delivery of a heart implant for treatment of mitral regurgitation.

FIG. 2A is an anatomic anterior view of a human heart, with portions broken away and in section to view the interior heart chambers and adjacent structures.

FIG. 2B is an anatomic superior view of a section of the human heart showing the tricuspid valve in the right atrium, the mitral valve in the left atrium, and the aortic valve in between, with the tricuspid and mitral valves open and the aortic and pulmonary valves closed during ventricular diastole (ventricular filling) of the cardiac cycle.

FIG. 2C is an anatomic superior view of a section of the human heart shown in FIG. 2B, with the tricuspid and mitral valves closed and the aortic and pulmonary valves opened during ventricular systole (ventricular emptying) of the cardiac cycle.

FIG. 2D is an anatomic anterior perspective view of the left and right atriums, with portions broken away and in section to show the interior of the heart chambers and associated structures, such as the fossa ovalis, coronary sinus, and the great cardiac vein.

FIG. 2E is a superior view of a healthy mitral valve, with the leaflets closed and coapting at peak contraction pressures during ventricular systole.

FIG. 2F is an anatomic superior view of a section of the human heart, with the normal mitral valve shown in FIG. 2E closed during ventricular systole (ventricular emptying) of the cardiac cycle.

FIG. 2G is a superior view of a dysfunctional mitral valve, with the leaflets failing to coapt during peak contraction pressures during ventricular systole, leading to mitral regurgitation.

FIG. 3 shows penetration of a vessel wall via the method of the invention and deployment of an anchor using a single catheter in one aspect of the invention.

FIG. 4 shows penetration of a vessel wall via the method of the invention and deployment of an anchor using a single catheter in one aspect of the invention.

FIG. 5 shows deployment of an anchor via the method of the invention using a single catheter in one aspect of the invention.

FIG. 6A is an anatomic anterior perspective view of the left and right atriums, with portions broken away and in section to show the presence of an implant system with an inter-atrial bridging element that spans the mitral valve annulus between a posterior anchor positioned in the great cardiac vein and an anterior anchor within the inter-atrial septum, which is suitable for delivery using the method of the invention.

FIG. 6B is an anatomic anterior perspective view of the left and right atriums, with portions broken away and in section to show the presence of an implant system with an inter-atrial bridging element that spans the mitral valve annulus between a posterior anchor positioned in the great cardiac vein and an anterior anchor within the inter-atrial septum, which is suitable for delivery using the method of the invention.

FIG. 7A is a detailed view showing an anterior anchor deployed within the fossa ovalis of the inter-atrial septum and the posterior anchor deployed within the great cardiac vein.

FIG. 7B as a detailed view showing an anterior anchor deployed within the fossa ovalis of the inter-atrial septum and the posterior anchor deployed within the great cardiac vein.

FIG. 8A shows a detailed view of an example anterior anchor of the implant that is suitable for anchoring within the patent fossa ovalis of the inter-atrial septum.

FIG. 8B shows a detailed view of an example anterior anchor of the implant that is suitable for anchoring within the patent fossa ovalis of the inter-atrial septum.

FIG. 9A shows an example locking bridge stop for locking the bridging element relative the anterior anchor of the implant.

FIG. 9B shows an example locking bridge stop for locking the bridging element relative the anterior anchor of the implant.

FIG. 10A shows an alternative example of a heart implant suitable for intravascular delivery in accordance with aspects of the invention.

FIG. 10B shows an alternative example of a heart implant suitable for intravascular delivery in accordance with aspects of the invention.

FIG. 11A shows an alternative example of posterior anchors attached to a bridging element for an implant suitable for intravascular delivery in accordance with aspects of the invention.

FIG. 11B shows an alternative example of posterior anchors attached to a bridging element for an implant suitable for intravascular delivery in accordance with aspects of the invention.

FIG. 12A shows an alternative example of a posterior anchor for a heart implant suitable for intravascular delivery in accordance with aspects of the invention.

FIG. 12B shows an alternative example of a posterior anchor for a heart implant suitable for intravascular delivery in accordance with aspects of the invention.

FIG. 13 shows penetration of a vessel wall via the method of the invention via deployment of a penetrating guidewire (e.g., crossing wire), using a catheter having a distally disposed stabilizing element (e.g., expandable balloon), and radiopaque marker in one aspect of the invention.

### DETAILED DESCRIPTION OF THE INVENTION

As discussed herein, the present invention provides methods for traversing an anatomical vessel wall of a subject. While the disclosure illustrates crossing of a cardiac vessel wall, such as the GCV into the left atrium, it will be appreciated that the methodology of the invention may be utilized in procedures involving any anatomical vessel to achieve crossing of a wire from one anatomical lumen, such as an artery, vein, esophagus, intestine or airway, through tissue, into another anatomical lumen, or cavity, or into a solid mass of tissue.

To achieve vessel wall crossing, conventional techniques require a catheter in one lumen and another catheter in the adjacent cavity to physically engage each, such as by magnetic attraction. The wire is advanced from one catheter, through the tissue wall, into the other catheter.

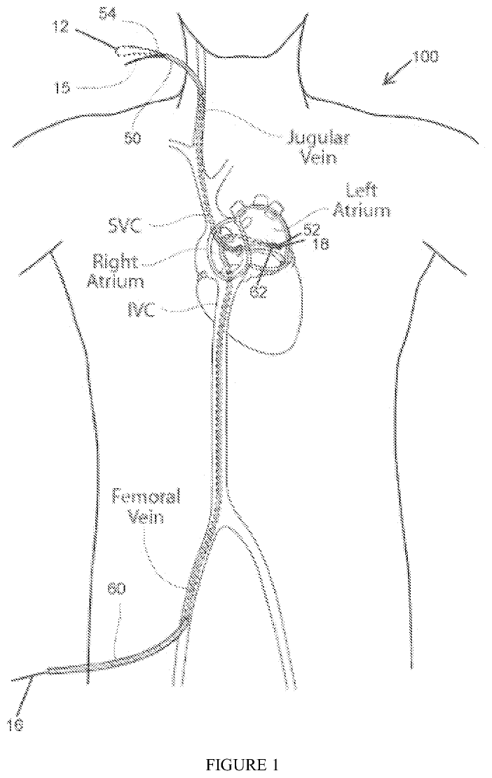

FIG. 1 shows an example of a conventional catheter-based delivery system which is used to reshape a heart chamber in the treatment of mitral valve regurgitation. The delivery system utilizes a pair of magnetic catheters that are advanced from separate vascular access points and magnetically coupled across a tissue within the heart. The pair of catheters include a great cardiac vein (GCV) anchor delivery catheter 50 which is introduced from the jugular vein and advanced along a superior vena cava (SVC) approach to the GCV, and a left atrial (LA) catheter 60, which is introduced at the femoral vein and introduced along an inferior vena cava (IVC) approach, across the inter-atrial septum and into the left atrium. Each catheter includes a magnetic head along a distal portion thereof (magnetic head 52 of catheter 50 and magnetic head 62 of catheter 60) such that when magnetically coupled, the catheters provide a stable region to facilitate penetration of a tissue wall between the LA and GCV and subsequent advancement of the puncturing guidewire 54 through the GCV catheter 50 and into the LA catheter 60. A trailing end of the puncturing guidewire 54 is attached to one end of a bridging element 12 (for example, suture), the other end of which is attached to posterior anchor 18 disposed on the distal portion of GCV catheter 50. Such a configuration allows the bridging element 12 to be advanced across the left atrium by advancing the puncturing guidewire 54 through the LA catheter 60 to exit from the femoral vein, while the magnetic heads remain magnetically coupled to each other.

Unlike conventional catheter systems and procedures, the present invention requires only one catheter to achieve vessel wall crossing. Use of a single catheter to achieve vessel wall crossing lowers costs associated with materials and components, as well as simplifying surgical procedures.

Accordingly, in one embodiment, the invention provides a method for traversing an anatomical vessel wall. The method includes advancing a catheter into a first anatomical lumen having a vessel wall to a first location. Once the catheter is advanced into the anatomical lumen to a desired position, the catheter is stabilized within the lumen using a stabilizing element.

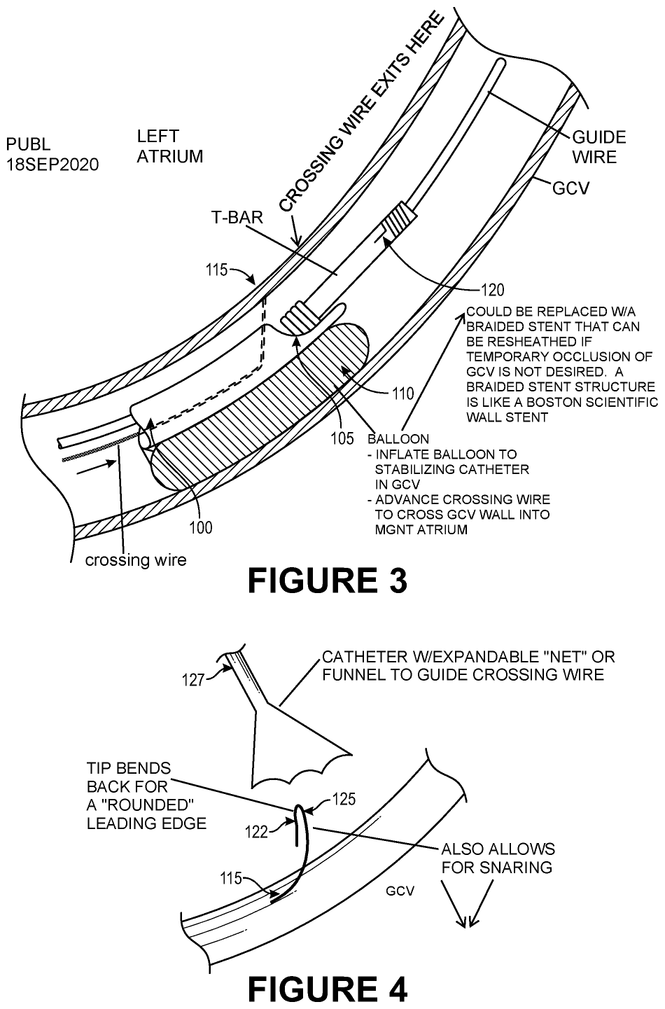

As such, in various aspects, the catheter 100 includes a lumen extending along a length of the catheter, a distally disposed opening 105, and a stabilizing element 110 as shown in FIG. 3. In some aspects, the stabilizing element 110 is disposed distally on the length of the catheter such that the stabilizing element 110 is positioned adjacent the distal catheter opening 105 when the stabilizing element is deployed.

FIG. 3 shows a catheter 100 disposed within the GCV and positioned adjacent the left atrium. The catheter 100 includes a stabilizing element 110 configured as an expandable balloon. Inflation of the balloon once the catheter opening is moved to the desired position within the GCV stabilizes the location of the balloon within the GCV to prevent movement of the catheter 100 while the vessel wall is punctured and one or more anchors 120 are deployed. In one aspect, the stabilizing element 110 is an expandable balloon. In another aspect, the stabilizing element 110 is an expandable stent. In some aspects, the stent is composed of a braid or mesh so as not to occlude blood flow through the vessel once the stent is deployed to stabilize the catheter.

Once the stabilizing element 110 is deployed, the penetrating guidewire 115 is advanced along the lumen of the catheter toward the distally disposed opening 105. As shown in FIG. 3, the penetrating guidewire 115, also referred to as a crossing wire, is advanced out of the distally disposed opening 105 and traverses the vessel wall into an adjacent anatomical lumen, such as the left atrium.

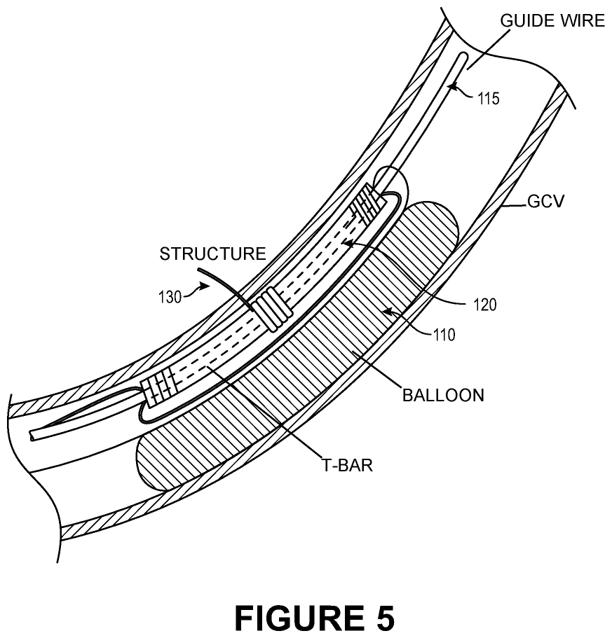

As discussed herein, the method of the invention may further include advancing an anchor 120, shown as a T-bar anchor in FIG. 3, via the catheter 100 to the site where the vessel wall is penetrated. As discussed herein, the anchor 120 may be part of an implant structure that is used to alter the shape of the heart chamber, e.g., the left atrium, via a bridging element 130 as shown in FIG. 5. In one aspect, the bridging element 130 is coupled to the anchor 120 via a first end and extends across the heart chamber to a second anchor deployed within or proximate the heart chamber at a second location as discussed further herein.

As shown in FIG. 4, the penetrating guidewire 115 may include a tip 122 that is composed of a shape memory material which forms a capture structure 125, such as a loop or hook once the tip 122 traverses the vessel wall. This allows the guidewire 115 to be captured, such as via a second guidewire or catheter to allow placement and/or deployment of one or more additional anchors of the implant. As shown in FIG. 4, the tip curls back on the guidewire 115 to form the capture structure 125 which may be snared or advanced into the lumen of a catheter. FIG. 4 shows a catheter 127 having a flared or funnel shaped distal end which allows the capture structure 125 to be guided into the lumen of the catheter 127.

As discussed herein, the penetrating guidewire includes a tip that is composed of a shape memory material. This allows the tip of the guidewire to be advanced along the GCV and across the vessel wall in a first generally straight configuration and then transition to a second bent configuration forming the capture structure. In some aspects, the tip forms a hook or V-shape in the second configuration. In some aspects, the tip forms a loop shape in the second configuration. In various aspects, the capture structure includes a bent or arcuate section that forms an angle of at least or greater than about 90, 100, 110, 120, 130, 140, 150, 160, 170, 180 or 190 degrees which allows the capture structure to be snared and pulled into the vessel cavity or into the lumen of a second catheter. In various aspects, the shape memory material is composed of a shape memory metal, alloy or plastic. In some aspects, the shape memory material is composed of a nickel-titanium (NiTi) or copper-aluminum-nickel alloy.

As discussed herein, the method and devices described herein are particularly useful for treatment of mitral valve regurgitation by reshaping a chamber of the heart, for example by reshaping the left atrium. As such, the invention also provides a method of treating mitral valve regurgitation in a subject by reshaping a heart chamber of a subject. The method includes inserting, through a vascular access site, a catheter, and advancing the catheter along a first anatomical lumen having a vessel wall to a first location proximate a heart of the subject, the catheter having a lumen extending along a length of the catheter, a distally disposed opening, and a stabilizing element; stabilizing the catheter within the first lumen via the stabilizing element at the first location; advancing a penetrating guidewire along the lumen of the catheter toward the distally disposed opening to the first location; penetrating the vessel wall by advancing the penetrating guidewire out of the distally disposed opening and traversing the vessel wall into a heart chamber; advancing a first anchor to the first location via the lumen of the catheter, wherein the first anchor is coupled to the first anchor at a first end of the bridging element; advancing a second end of the bridging element through the penetrated vessel wall at the first location; advancing a second anchor along the bridging element and deploying the second anchor at a second location in or proximate the heart, the bridging element spanning across the heart chamber; and shortening a length of the bridging element thereby reshaping the chamber of the heart and coupling the second end of the bridging element to the deployed second anchor while the chamber of the heart is reshaped so that the chamber of the heart remains reshaped, thereby treating mitral valve regurgitation in the subject.

FIG. 5 illustrates deployment of an anchor within the GCV. As shown, the bridging element 130 of the anchor 120 traverses the tissue wall via the hole formed by a penetrating guidewire deployed from a catheter while the stabilizing element is deployed. In some aspects, withdrawal of the guidewire 115 causes the anchor 120 to detach from the guidewire 115 and remain in the GCV such that it may be coupled, via the bridging element 130 to one or more additional anchors disposed within or proximate the heart chamber in another location.

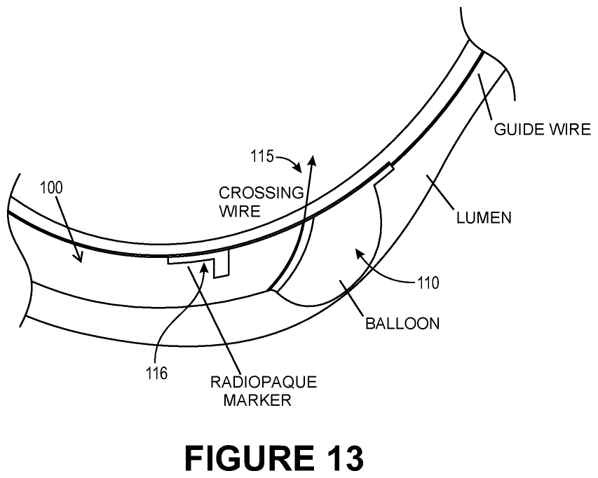

FIG. 13 shows penetration of a vessel wall via the method of the invention via deployment of a penetrating guidewire 115, using a catheter 100 having a distally disposed stabilizing element 110, and radiopaque marker 116 in one aspect of the invention. In various aspects, the catheter 100 includes a pre-curved shaft that mimics curvature of a heart surface or anatomical vessel, such as a coronary sinus, GCV or the like. In various aspects, the radiopaque marker 116 has a unique shape that indicates specific orientation and placement of the stabilizing element 110 to the user for proper placement during a procedure and crossing of the penetrating guidewire 115 across the vessel wall.

Heart Implants for Treatment/Repair of a Heart Valve Annulus

Illustrative Implant Structures for Use with the Invention

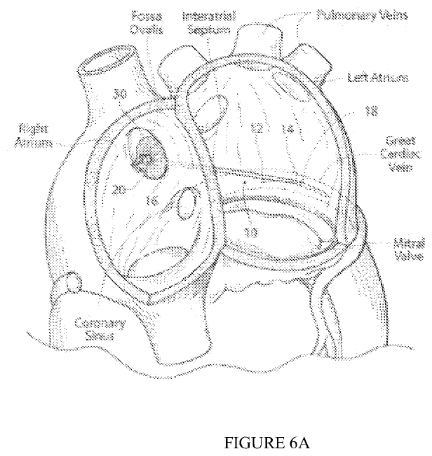

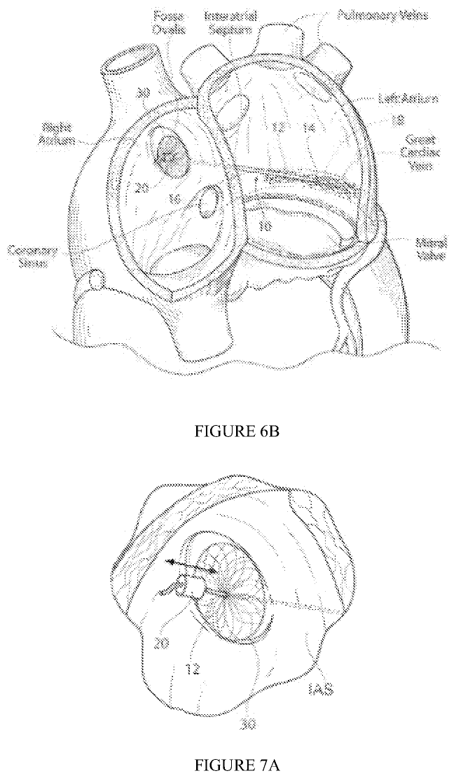

FIGS. 6A-6B show embodiments of an implant 10 that is sized and configured to extend across the left atrium in generally an anterior-to-posterior direction, spanning the mitral valve annulus. The implant 10 comprises a spanning region or bridging element 12 having a posterior anchor region 14 and an anterior anchor region 16.

The posterior anchor region 14 is sized and configured to allow the bridging element 12 to be placed in a region of atrial tissue above the posterior mitral valve annulus. This region is preferred, because it generally presents more tissue mass for obtaining purchase of the posterior anchor region 14 than in a tissue region at or adjacent to the posterior mitral annulus. Engagement of tissue at this supra-annular location also may reduce risk of injury to the circumflex coronary artery. In a small percentage of cases, the circumflex coronary artery may pass over and medial to the great cardiac vein on the left atrial aspect of the great cardiac vein, coming to lie between the great cardiac vein and endocardium of the left atrium. However, since the forces in the posterior anchor region are directed upward and inward relative to the left atrium and not in a constricting manner along the long axis of the great cardiac vein, the likelihood of circumflex artery compression is less compared to other technologies in this field that do constrict the tissue of the great cardiac vein. Nevertheless, should a coronary angiography reveal circumflex artery stenosis, the symmetrically shaped posterior anchor may be replaced by an asymmetrically shaped anchor, such as where one limb of a T-shaped member is shorter than the other, thus avoiding compression of the crossing point of the circumflex artery. The asymmetric form may also be selected first based on a pre-placement angiogram.

An asymmetric posterior anchor may be utilized for other reasons as well. The asymmetric posterior anchor may be selected where a patient is found to have a severely stenotic distal great cardiac vein, where the asymmetric anchor better serves to avoid obstruction of that vessel. In addition, an asymmetric anchor may be chosen for its use in selecting application of forces differentially and preferentially on different points along the posterior mitral annulus to optimize treatment, for example, in cases of malformed or asymmetrical mitral valves.

The anterior anchor region 16 is sized and configured to allow the bridging element 12 to be placed, upon passing into the right atrium through the septum, adjacent tissue in or near the right atrium. For example, as is shown in FIGS. 6A-6B, the anterior anchor region 16 may be adjacent or abutting a region of fibrous tissue in the interatrial septum. As shown, the anchor site 16 is desirably superior to the anterior mitral annulus at about the same elevation or higher than the elevation of the posterior anchor region 14. In the illustrated embodiment, the anterior anchor region 16 is adjacent to or near the inferior rim of the fossa ovalis. Alternatively, the anterior anchor region 16 can be located at a more superior position in the septum, for example, at or near the superior rim of the fossa ovalis. The anterior anchor region 16 can also be located in a more superior or inferior position in the septum, away from the fossa ovalis, provided that the anchor site does not harm the tissue in the region.

Alternatively, the anterior anchor region 16, upon passing through the septum into the right atrium, may be positioned within or otherwise extend to one or more additional anchors situated in surrounding tissues or along surrounding areas, such as within the superior vena cava (SVC) or the inferior vena cava (IVC).

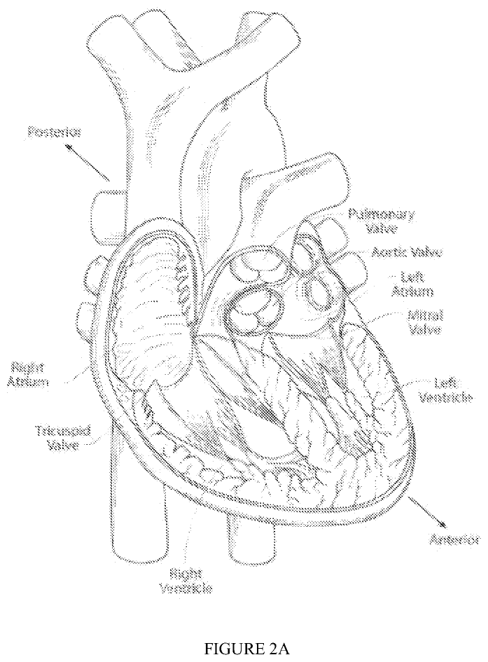

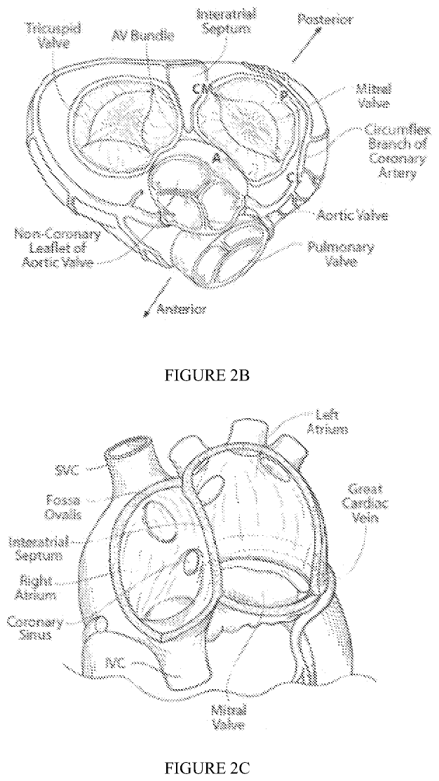

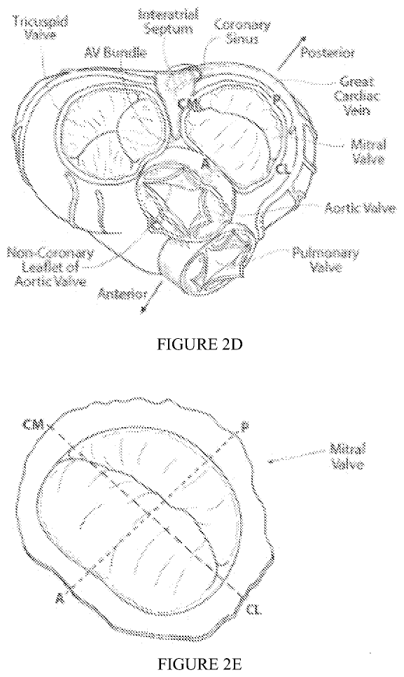

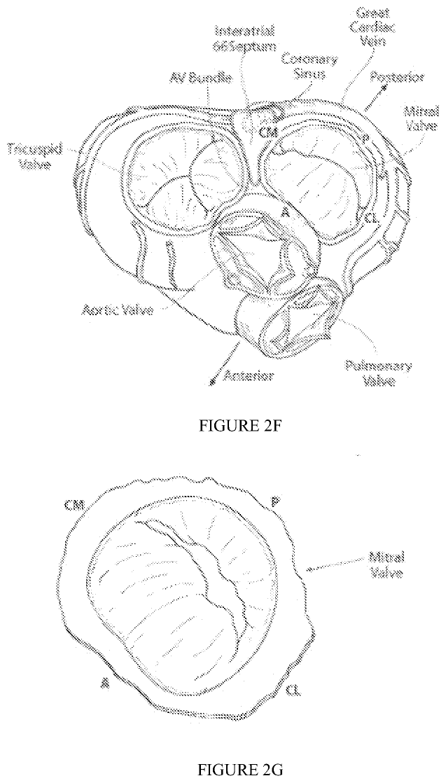

In use, the spanning region or bridging element 12 can be placed into tension between the two anchor regions 14 and 16. The implant 10 thereby serves to apply a direct mechanical force generally in a posterior to anterior direction across the left atrium. The direct mechanical force can serve to shorten the minor axis (along line P-A in FIG. 2E) of the annulus. In doing so, the implant 10 can also reactively reshape the annulus along its major axis (line CM-CL in FIG. 2E) and/or reactively reshape other surrounding anatomic structures. It should be appreciated, however, the presence of the implant 10 can serve to stabilize tissue adjacent the heart valve annulus, without affecting the length of the minor or major axes.

It should also be appreciated that, when situated in other valve structures, the axes affected may not be the “major” and “minor” axes, due to the surrounding anatomy. In addition, in order to be therapeutic, the implant 10 may only need to reshape the annulus during a portion of the heart cycle, such as during late diastole and early systole when the heart is most full of blood at the onset of ventricular systolic contraction, when most of the mitral valve leakage occurs. For example, the implant 10 may be sized to restrict outward displacement of the annulus during late ventricular diastolic relaxation as the annulus dilates.

The mechanical force applied by the implant 10 across the left atrium can restore to the heart valve annulus and leaflets a more normal anatomic shape and tension. The more normal anatomic shape and tension are conducive to coaptation of the leaflets during late ventricular diastole and early ventricular systole, which, in turn, reduces mitral regurgitation.

In its most basic form, the implant 10 is made from a biocompatible metallic or polymer material, or a metallic or polymer material that is suitably coated, impregnated, or otherwise treated with a material to impart biocompatibility, or a combination of such materials. The material is also desirably radio-opaque or incorporates radio-opaque features to facilitate fluoroscopic visualization.

In some embodiments, the implant 10, or at least a portion thereof, can be formed by bending, shaping, joining, machining, molding, or extrusion of a metallic or polymer wire form structure, which can have flexible or rigid, or inelastic or elastic mechanical properties, or combinations thereof. In other embodiments, the implant 10, or at least a portion thereof, can be formed from metallic or polymer thread-like or suture material. Materials from which the implant 10 can be formed include, but are not limited to, stainless steel, Nitinol, titanium, silicone, plated metals, Elgiloy™, NP55, and NP57.

In any of the implants described herein, the bridging member can be formed of a substantially inelastic material, such as a thread-like or suture material.

The Posterior Anchor Region

The posterior anchor region 14 is sized and configured to be located within or at the left atrium at a supra-annular position, for example, positioned within or near the left atrium wall above the posterior mitral annulus.

In the illustrated embodiment, the posterior anchor region 14 is shown to be located generally at the level of the great cardiac vein, which travels adjacent to and parallel to the majority of the posterior mitral valve annulus. This extension of the coronary sinus can provide a strong and reliable fluoroscopic landmark when a radio-opaque device is placed within it or contrast dye is injected into it. As previously described, securing the bridging element 12 at this supra-annular location also lessens the risk of encroachment of and risk of injury to the circumflex coronary artery compared to procedures applied to the mitral annulus directly. Furthermore, the supra-annular position assures no contact with the valve leaflets therefore allowing for coaptation and reduces the risk of mechanical damage.

The great cardiac vein also provides a site where relatively thin, non-fibrous atrial tissue can be readily augmented and consolidated. To enhance hold or purchase of the posterior anchor region 14 in what is essentially non-fibrous heart tissue, and to improve distribution of the forces applied by the implant 10, the posterior anchor region 14 may include a posterior anchor 18 placed within the great cardiac vein and abutting venous tissue. This makes possible the securing of the posterior anchor region 14 in a non-fibrous portion of the heart in a manner that can nevertheless sustain appreciable hold or purchase on that tissue for a substantial period of time, without dehiscence, expressed in a clinically relevant timeframe.

The Anterior Anchor Region

The anterior anchor region is sized and configured to allow the bridging element 12 to remain firmly in position adjacent or near the fibrous tissue and the surrounding tissues in the right atrium side of the atrial septum. The fibrous tissue in this region provides superior mechanical strength and integrity compared with muscle and can better resist a device pulling through. The septum is the most fibrous tissue structure in its own extent in the heart.

Surgically handled, it is usually one of the only heart tissues into which sutures actually can be placed and can be expected to hold without pledgets or deep grasps into muscle tissue, where the latter are required.

As shown in FIGS. 6A-6B, the anterior anchor region 16 passes through the septal wall at a supra-annular location above the plane of the anterior mitral valve annulus. The supra-annular distance on the anterior side can be generally at or above the supra-annular distance on the posterior side. The anterior anchor region 16 is shown at or near the inferior rim of the fossa ovalis, although other more inferior or more superior sites can be used within or outside the fossa ovalis, taking into account the need to prevent harm to the septal tissue and surrounding structures.

By locating the bridging element 12 at this supra-annular level within the right atrium, which is fully outside the left atrium and spaced well above the anterior mitral annulus, the implant 10 avoids the impracticalities of endovascular attachment at or adjacent to the anterior mitral annulus, where there is just a very thin rim of annulus tissue that is bounded anteriorly by the anterior leaflet, inferiorly by the aortic outflow tract, and medially by the atrioventricular node of the conduction system. The anterior mitral annulus is where the non-coronary leaflet of the aortic valve attaches to the mitral annulus through the central fibrous body. Anterior location of the implant 10 in the supra-annular level within the right atrium (either in the septum or in a vena cava) avoids encroachment of and risk of injury to both the aortic valve and the AV node.

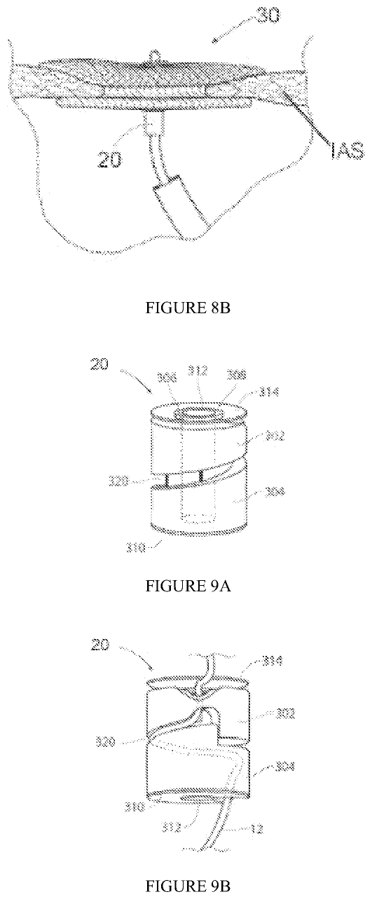

The purchase of the anterior anchor region 16 in fibrous septal tissue is desirably enhanced by a septal member 30 or an anterior anchor 20, or a combination of both. FIGS. 8A and 8B show the anterior anchor region including a septal member 30. The septal member 30 may be an expandable device and also may be a commercially available device such as a septal occluder, for example, Amplatzer® PFO Occluder. The septal member 30 preferably mechanically amplifies the hold or purchase of the anterior anchor region 16 in the fibrous tissue site. The septal member 30 also desirably increases reliance, at least partly, on neighboring anatomic structures of the septum to make firm the position of the implant 10. In addition, the septal member 30 may also serve to plug or occlude the small aperture that was created in the fossa ovalis or surrounding area during the implantation procedure.

Anticipating that pinpoint pulling forces will be applied by the anterior anchor region 16 to the septum, the forces acting on the septal member 30 should be spread over a moderate area, without causing impingement on valve, vessels or conduction tissues. With the pulling or tensioning forces being transmitted down to the annulus, shortening of the minor axis is achieved. A flexurally stiff septal member is preferred because it will tend to cause less focal narrowing in the direction of bridge element tension of the left atrium as tension on the bridging element is increased. The septal member 30 should also have a low profile configuration and highly washable surfaces to diminish thrombus formation for devices deployed inside the heart. The septal member may also have a collapsed configuration and a deployed configuration. The septal member 30 may also include a hub 31 (see FIGS. 8A and 8B) to allow attachment of the anchor 20. A septal brace may also be used in combination with the septal member 30 and anterior anchor 20 to distribute forces uniformly along the septum. Alternatively, devices in the IVC or the SVC can be used as anchor sites, instead of confined to the septum.

Location of the posterior and anterior anchor regions 14 and 16 having radio-opaque bridge locks and well demarcated fluoroscopic landmarks respectively at the supra-annular tissue sites just described, not only provides freedom from key vital structure damage or local impingement, for example, to the circumflex artery, AV node, and the left coronary and noncoronary cusps of the aortic valve; but the supra-annular focused sites are also not reliant on purchase between tissue and direct tension-loaded penetrating/biting/holding tissue attachment mechanisms. Instead, physical structures and force distribution mechanisms such as stents, T-shaped members, and septal members can be used, which better accommodate the attachment or abutment of mechanical levers and bridge locks, and through which potential tissue tearing forces can be better distributed. Further, the anchor sites 14, 16 do not require the operator to use complex imaging. Adjustment of implant position after or during implantation is also facilitated, free of these constraints. The anchor sites 14, 16 also make possible full intra-atrial retrieval of the implant 10 by endovascularly snaring and then cutting the bridging element 12 at either side of the left atrial wall, from which it emerges.

Orientation of the Bridging Element

In the embodiments shown in FIGS. 6A-6B, the implant 10 is shown to span the left atrium beginning at a posterior point of focus superior to the approximate mid-point of the mitral valve annulus, and proceeding in an anterior direction in a generally straight path directly to the region of anterior focus in the septum. The spanning region or bridging element 12 of the implant 10 may be preformed or otherwise configured to extend in this essentially straight path above the plane of the valve, without significant deviation in elevation toward or away from the plane of the annulus, other than as dictated by any difference in elevation between the posterior and anterior regions of placement. It is appreciated that such implants can include bridging member with lateral or medial deviations and/or superior or inferior deviations and can include bridging members that are rigid or semi-rigid and/or substantially fixed in length.

Posterior and Anterior Anchors

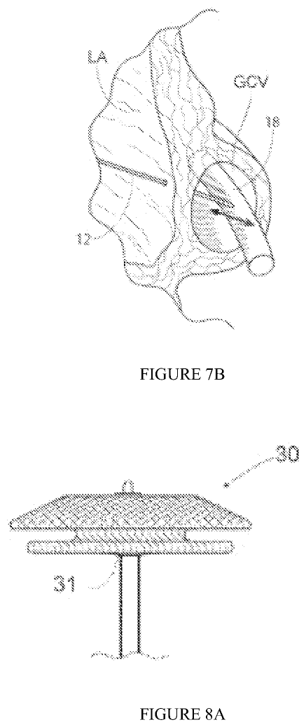

It is to be appreciated that an anchor as described herein, including a posterior or anterior anchor, describes an apparatus that may releasably hold the bridging element 12 in a tensioned state. As can be seen in FIGS. 7A-7B, anchors 20 and 18 respectively are shown releasably secured to the bridging element 12, allowing the anchor structure to move back and forth independent of the inter-atrial septum and inner wall of the great cardiac vein during a portion of the cardiac cycle when the tension force may be reduced or becomes zero.

Alternative embodiments are also described, all of which may provide this function. It is also to be appreciated that the general descriptions of posterior and anterior anchors are non-limiting to the anchor function, for example, a posterior anchor may be used anterior, and an anterior anchor may be used posterior.

When the bridging element is in an abutting relationship to a septal member (for example, anterior anchor) or a T-shaped member (for example, posterior anchor), for example, the anchor allows the bridging element to move freely within or around the septal member or T-shaped member, for example, the bridging element is not connected to the septal member or T-shaped member. In this configuration, the bridging element is held in tension by the locking bridge stop, whereby the septal member or T-shaped member serves to distribute the force applied by the bridging element across a larger surface area. Alternatively, the anchor may be mechanically connected to the septal member or T-shaped member, for example, when the bridge stop is positioned over and secured to the septal member hub. In this configuration, the bridging element is fixed relative to the septal member position and is not free to move about the septal member.

FIGS. 9A-9B show perspectives views of an example locking bridge stop 20 in accordance with the present invention. Each bridge stop 20 preferably includes a fixed upper body 302 and a movable lower body 304. Alternatively, the upper body 302 may be movable and the lower body 304 may be fixed. The upper body 302 and lower body 304 are positioned circumjacent a tubular shaped rivet 306. The upper body 302 and lower body 304 are preferably held in position by the rivet head 308 and a base plate 310. The rivet 306 and base plate 310 includes a predetermined inner diameter 312, sized so as to allow the bridge stop 300 to be installed over a guidewire. A spring, such as a spring washer 314, or also known in the mechanical art as a Belleville Spring, is positioned circumjacent the rivet 306 and between the rivet head 308 and the upper body 302, and applies an upward force on the lower body 304. The lower body 304 is movable between a bridge unlocked position (see FIG. 9A), and a bridge locked position (see FIG. 9B). In the bridge unlocked position, the lower body 304 and the upper body 302 are not in contacting communication, creating a groove 320 between the upper body 302 and lower body 304. In the bridge locked position, the axial force of the spring washer 314 urges the lower body 304 into contacting, or near contacting communication with the upper body 302, whereby the bridging element 12, which has been positioned within the groove 320, is locked in place by the axial force of the lower body 304 being applied to the upper body 302. In use, the bridging element 12 is positioned within the groove 320 while the lower body 304 is maintained in the bridge unlocked position 316. The bridge stop 300 is positioned against the septal member 30 and the bridging element 12 is adjusted to proper tension. The lower body 304 is then allowed to move toward the upper body 302, thereby fixing the position of the bridge stop 300 on the bridging element 12. While this example depicts a particular locking bridge stop design, it is appreciated that any suitable lock could be used, including any of the types described in U.S. Patent Application Publication No. 2017/0055969.

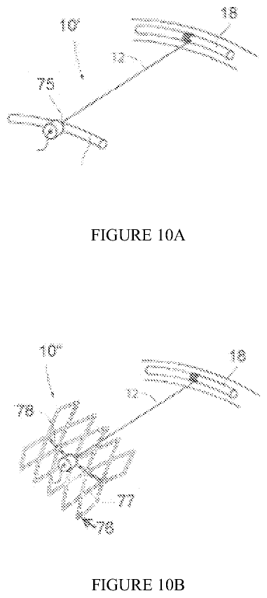

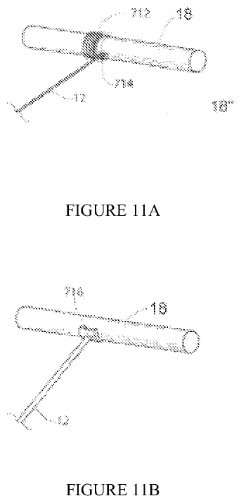

FIGS. 10A-10B show alternative heart implants suitable for delivery with the method described herein. FIG. 10A shows an implant 10′ having a T-shaped posterior anchor 18 in the great cardiac vein and T-shaped anterior anchor 70. The anterior T-shaped bridge stop 75 may be of a construction of any of the T-shaped bridge stop embodiments described. The T-shaped member 75 includes a lumen 75 extending through the T-shaped member 75 perpendicular to the length of the T-shaped member. The bridging element 12 may be secured by a free floating bridge stop as previously described. FIG. 10B shows an implant 10′ having a T-shaped posterior anchor 18 in the great cardiac vein and a lattice style anterior anchor 76. The lattice 77 is positioned on the septal wall at or near the fossa ovalis. Optionally, the lattice 77 may include a reinforcement strut 78 to distribute the bridging element 12 tension forces over a greater area on the septal wall. The anterior lattice style bridge stop 76 may be packed in a deployment catheter with the bridging element 12 passing through its center. The lattice 77 is preferably self-expanding and may be deployed by a plunger. The bridging element 12 may be secured by a free floating bridge stop as previously described. It is appreciated that various other such implants could be devised that utilized the same concepts as in the above described implants for delivery and deployment with the method described herein.

FIGS. 11A-11B show alternative methods of connecting the bridging element 12 to a T-shaped posterior anchor. FIG. 11A shows a T-shaped member 18 where the bridging element 12 is wound around a central portion of the T-shaped member. The bridging element 12 may be secured by adhesive 712, knot, or a securing band placed over the bridging element 12, for example. Alternatively, the bridging element 12 may first be threaded through a lumen 714 extending through the T-shaped posterior anchor 18 perpendicular the length of the T-shaped member. The bridging element 12 may then be wound around the T-shaped member, and secured by adhesive 712, securing band, or knot, for example. FIG. 11B shows a T-shaped member 18 where the bridging element 12 is welded or forged to a plate 716. The plate 716 may then be embedded within the T-shaped member 710, or alternatively, secured to the T-shaped member 710 by gluing or welding, for example. It is appreciated that various other couplings could be used to secure the bridging element 12 and posterior anchor 18 and facilitate delivery with the method described herein.

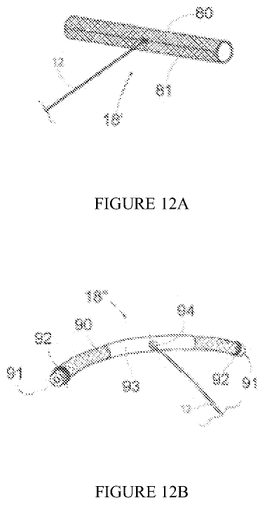

FIGS. 12A-12B depict alternative anchors suitable for use as posterior anchors within a heart implant in accordance with the invention. FIG. 12A is a perspective view of a T-shaped anchor 18′ that includes an intravascular stent 80 and, optionally, a reinforcing strut 81. The stent 80 may be a balloon expandable or self-expanding stent. As previously described, the T-shaped anchor 18′ is preferably connected to a predetermined length of the bridging element 12. The bridging element 12 may be held within, on, or around the T-shaped bridge stop 80 through the use of any of the bridge locks as previously described, or may be connected to the T-shaped anchor 18 by way of tying, welding, or gluing, for example, or any combination. FIG. 12B depicts a T-shaped anchor 18″ that includes a flexible tube 90 having a predetermined length, for example, three to eight centimeters, and an inner diameter 91 sized to allow at least a guidewire to pass through. The tube 90 is preferably braided, but may be solid as well, and may also be coated with a polymer material. Each end of the tube 90 preferably includes a radio-opaque marker 92 to aid in locating and positioning the T-shaped anchor. The tube 90 also preferably includes atraumatic ends to protect the vessel walls. The tube may be flexurally curved or preshaped so as to generally conform to the curved shape of the great cardiac vein or interatrial septum and be less traumatic to surrounding tissue. A reinforcing center tube 93 may also be included to add stiffness to the anchor and aids in preventing egress of the anchor from the great cardiac vein and left atrium wall. The bridging element 12 extends through a central hole 94 in an interior side of the reinforcing center tube 93. Each of the anchors described can be straight or curvilinear in shape, or flexile so as to accommodate an anatomy. It is appreciated that various other type of anchors could be used a posterior anchor 18 attached to bridging element 12 for delivery and deployment with the method described herein.

General Methods of Delivery and Implantation

The implant systems 10 described herein lend themselves to implantation in a heart valve annulus in various ways. In some aspects, the implants 10 are implanted using catheter-based technology via a peripheral venous access site, such as in the femoral or jugular vein (via the IVC or SVC) under image guidance, or trans-arterial retrograde approaches to the left atrium through the aorta from the femoral artery also under image guidance. As previously described, the implants 10 comprise independent components that are assembled within the body to form an implant, and delivered and assembled from an exterior to the body through interaction of a single or multiple catheters. However, penetration of heart tissue is performed via interactions with a single catheter.

Although the invention has been described with reference to the above examples, it will be understood that modifications and variations are encompassed within the spirit and scope of the invention. Accordingly, the invention is limited only by the following claims.

1. A method for traversing a vessel wall comprising:
advancing a catheter into a first anatomical lumen having a vessel wall to a first location, the catheter comprising a lumen extending along a length of the catheter, a distally disposed opening, and a stabilizing element;
stabilizing the catheter within the first lumen via the stabilizing element at the first location;
advancing a penetrating guidewire along the lumen of the catheter toward the distally disposed opening to the first location, wherein the penetrating guidewire comprises a tip, the tip having shape memory and configured to form a capture structure upon crossing the vessel wall; and
penetrating the vessel wall by advancing the penetrating guidewire out of the distally disposed opening and traversing the vessel wall into a second anatomical lumen or tissue, thereby traversing the vessel wall.

2. The method of claim 1, wherein the stabilizing element comprises an expandable balloon or stent.

3. The method of claim 1, wherein the capture structure comprises a hook or a loop structure, and optionally wherein the hoop or the loop comprises a bent section having an angle of greater than about 90, 100, 110, 120, 130, 140, 150, 160, 170, 180 or 190 degrees.

4. The method of claim 3, further comprising advancing a first anchor to the first location via the lumen of the catheter.

5. The method of claim 4, wherein the first anchor includes a bridging element coupled to the anchor at a first end of the bridging element.

6. The method of claim 5, further comprising advancing a second end of the bridging element through the penetrated vessel wall at the first location.

7. The method of claim 6, further comprising advancing a second anchor to a second location within or proximate the second lumen and deploying the second anchor at the second location, wherein the first anchor is coupled to the first end of the bridging element and the second anchor is coupled to the second end of the bridging element.

8. The method of claim 7, further comprising tensioning the bridging element.

9. The method of claim 8, wherein the first location is proximate a heart chamber.

10. The method of claim 9, wherein the second location is within or proximate the heart chamber.

11. The method of claim 10, wherein the heart chamber is the left atrium and the first location is within a great cardiac vein.

12. The method of claim 10, wherein the bridging element spans the heart chamber and tensioning of the bridging element reshapes the heart chamber.

13. The method of claim 2, further comprising coupling a guidewire to the capture structure.

14. The method of claim 4, wherein the first anchor is advanced to the first location via a guidewire.

15. The method of claim 14, further comprising releasing the first anchor from the guidewire by withdrawing the guidewire along the lumen of the catheter.

16. The method of claim 1, further comprising determining the depth of insertion of the catheter into the first lumen to determine the first position.

17. A method of treating mitral valve regurgitation in a subject comprising:
inserting, through a vascular access site, a catheter, and advancing the catheter along a first anatomical lumen having a vessel wall to a first location proximate a heart of the subject, the catheter comprising a lumen extending along a length of the catheter, a distally disposed opening, and a stabilizing element;
stabilizing the catheter within the first lumen via the stabilizing element at the first location;
advancing a penetrating guidewire along the lumen of the catheter toward the distally disposed opening to the first location;
penetrating the vessel wall by advancing the penetrating guidewire out of the distally disposed opening and traversing the vessel wall into a heart chamber, wherein the penetrating guidewire comprises a tip, the tip having shape memory and configured to form a capture structure upon crossing the vessel wall;
advancing a first anchor to the first location via the lumen of the catheter, wherein the first anchor is coupled to the first anchor at a first end of the bridging element;
advancing a second end of the bridging element through the penetrated vessel wall at the first location;
advancing a second anchor along the bridging element and deploying the second anchor at a second location in or proximate the heart, the bridging element spanning across the heart chamber; and
shortening a length of the bridging element thereby reshaping the chamber of the heart and coupling the second end of the bridging element to the deployed second anchor while the chamber of the heart is reshaped so that the chamber of the heart remains reshaped, thereby treating mitral valve regurgitation in the subject.

18. The method of claim 17, wherein the stabilizing element comprises an expandable balloon or stent.

19. The method of claim 17, wherein the capture structure comprises a hook or a loop structure, and optionally wherein the hoop or the loop comprises a bent section having an angle of greater than about 90, 100, 110, 120, 130, 140, 150, 160, 170, 180 or 190 degrees.

20. The method of claim 17, wherein the heart chamber is the left atrium and the first location is within a great cardiac vein.

21. The method of claim 17, wherein the first anchor is advanced to the first location via a guidewire.

22. The method of claim 21, further comprising releasing the first anchor from the guidewire by withdrawing the guidewire along the lumen of the catheter.

23. The method of claim 17, further comprising determining the depth of insertion of the catheter into the first lumen to determine the first position.
